(19) United States
(12) Patent Application Publication
(10) Pub. No.: US 2022/0040124 A1
(43) Pub. Date: Feb. 10, 2022

(54) COMPOSITIONS COMPRISING CBD FOR TREATING MENTAL DISORDERS

(71) Applicant: UNV MEDICINE LTD., Ashkelon (IL)

(72) Inventors: Omer LAYANI, Ashkelon (IL); Ilana LAVON, Ashkelon (IL); Shaul MUKHTAR, Ashkelon (IL)

(21) Appl. No.: 17/281,364

(22) PCT Filed: Sep. 26, 2019
(86) PCT No.: PCT/IL2019/051066

§ 371 Date: Mar. 30, 2021

(30) Foreign Application Priority Data
Oct. 2, 2018 (IL) 262049

(51) Int. Cl. A61K 31/05; A61K 36/68; A61K 36/23; A61K 36/81; A61P 25/00

(52) U.S. Cl. CPC A61K 31/05; A61K 36/68; A61K 36/23; A61K 36/81; A61K 2236/37; A61K 2236/333; A61K 2236/53; A61K 2236/51; A61K 2236/15; A61P 25/00

(57) ABSTRACT

The present invention discloses compositions comprising CBD and several herbal extract and use thereof in treatment of mental disorders such as Attention Deficit Hyperactivity disorder (ADHD) and Post Traumatic Stress Disorder (PTSD).

### FIELD OF THE INVENTION

The present invention relates to compositions and methods for treatment and management of mental disorders such as Attention Deficit Hyperactivity disorder (ADHD) and Post Traumatic Stress Disorder (PTSD).

### BACKGROUND OF THE INVENTION

Attention-deficit hyperactivity disorder (ADHD) is a mental disorder of the neurodevelopmental type. It is characterized by problems paying attention, excessive activity, or difficulty controlling behavior. ADHD is divided into three main subtypes: predominantly inattentive (ADHD-PI or ADHD-I), predominantly hyperactive-impulsive (ADHD-PH or ADHD-HI), and combined type (ADHD-C). ADHD may be diagnosed by various criteria, such as the DSM-IV criteria, by which 5-7% of children are diagnosed as having ADHD, or the ICD-10 criteria, by which 1-2% of children are diagnosed as having ADHD.

Medical treatment of ADHD includes stimulant medications as a pharmaceutical treatment, which exhibit at least some effect on symptoms, in the short term, in about 80% of people. Methylphenidate appears to improve the symptoms mentioned, and stimulants as a whole may reduce the risk of unintentional injuries in children with ADHD. However, stimulant use has also been associated with side effects including tremor, insomnia, nausea, lack of appetite and the like.

There are a number of non-stimulant medications, such as atomoxetine, bupropion, guanfacine, and clonidine that may be used as alternatives, or added to stimulant therapy.

Traditional medicine, including use of plants, has been implicated in the treatment of neuropsychopharmacology conditions. For example, Dua J. S. et. al. discusses the role of traditional medicine in neuropsychopharmacology (Asian Journal of pharmaceutical and clinical research, 2009, Vol. 2, pages 72-76). For example, a review by Kumar G. P. and Khanum F, discusses the neuroprotective potential of phytochemicals (Pharmacogn Rev., 2012, 6(12), 81-90).

WO 03/068251 describes herbal compositions comprising Bacopa monnieri, Centella Asiatica, Glycyrrhiz glabra, Withania Somnifera and Celastrus paniculaus oil for treating ADHD. Additional herbal compositions, some of which comprise CBD, comprising different content of herbal extracts were also described as allegedly effective in treating ADHD.

Post-traumatic stress disorder (PTSD) results from experiencing or witnessing a traumatic event that causes intense fear, helplessness or horror. It results in symptoms that fall into three types: re-experiencing the event, emotional numbing and avoidance and hyperarousal. Repetition of these overwhelming emotions can lead to a cascade of biological events including excessive release of epinephrine and norepinephrine which overpowers the autonomic response leading to clamminess, increased heart rate and breathing, increased blood flow to the muscles and decreased blood flow to the visceral organs. It is currently theorized that this response leads to deep imprinting on the locus coeruleus region of the brain and makes it over sensitized to any further threats (real or imaginary). (Diagnostic and Statistical Manual of Mental Disorders 4th edition (DSM-IV) published by the American Psychiatric Association (APA; Washington, D.C., 1994)). PTSD, as that term is used herein, also encompasses the 4 categories of symptoms outlined in the Diagnostic and Statistical Manual of Mental Disorders 5^{th} Edition (DSM-V) published by the American Psychiatric Association (APA; Washington, D.C., 2013), which include intrusion (e.g., re-experiencing), avoidance, negative alterations in mood and cognition and alterations in arousal and reactivity.

Exposure to traumatic events is common with more than 50% of the US population experiencing one or more traumatic events in their lifetime. However, rates of PTSD vary according to population, with a lifetime prevalence of approximately 5 to 12% of the population, with women having twice the prevalence rate of men and certain segments of the population, such as combat soldiers having rates as high as 25%.

The Cannabis plant has long been used for medicinal and recreational purposes alike. Subsequently, this plant has been characterized as containing many naturally occurring substances of great interest in the fields of science and medicine. The medical use of Cannabis is now legal in many countries under specific “medical Cannabis” legislation. Medical Cannabis is used for treating and alleviating symptoms associated with a growing number of indications, including pain, anorexia, asthma, glaucoma, arthritis, spasms, anxiety, and substance withdrawal. Many other illnesses are emerging as potential Cannabis-responsive indications, including various neuropsychological conditions, including autism.

There remains a need for safe and efficient compositions for the treatment and management of mental disorders such as ADHD and PTSD, which are effective, natural and have reduced side effects.

### SUMMARY OF THE INVENTION

The present invention in embodiments thereof relates to compositions and methods for the treatment of mental disorders such as ADHD and PTSD, wherein the compositions include Cannabis extract. In particular, embodiments of the invention provide compositions useful in the treatment of ADHD and PTSD and amelioration of symptoms associated therewith.

The invention is based, in part on the development of formulations (compositions), having unexpectedly advantageous properties in the treatment of ADHD and PTSD. In various embodiments, the compositions were found to exhibit marked and consistent efficacy and safety in the treatment of ADHD and/or symptoms associated therewith, while being efficient and having reduced side effects. According to other embodiments, the compositions were found to exhibit enhanced absorption, tolerability, and efficiency, as disclosed herein.

In particular, it was discovered that compositions comprising oil extracts from cannabidiol (CBD)-rich (CBD-dominant) Cannabis strains were more effective in alleviating ADHD and PTSD than compositions comprising tetrahydrocannabinol (THC)-dominant strains.

The compositions of the invention comprise a unique combination of ingredients from Cannabis and other botanical sources. This unique combination provides advantageous properties over the use of each ingredient alone, which may be in various embodiments additive or greater than additive (synergistic), depending on the particular measured outcome.

According to the teaching of the invention, the composition comprises extracts obtained from Cannabis (Cannabis Sativa L., e.g. Cannabis sativa and/or Cannabis indica strains approved for medical use).

According to one aspect, the present invention provides a composition comprising from about 1 to about 30% by weight of CBD oil, from about 6 to about 60% by weight of Bacopa monnieri extract, from about 6 to about 60% by weight of Centella Asiatica extract, and from about 5 to about 60% by weight of Withania Somnifera extract.

According to a typical embodiment of the invention, the composition comprises CBD oil, namely a full-plant Cannabis oil extraction, typically from cannabidiol (CBD)-dominant strains. An exemplary CBD-dominant Cannabis strain comprises about 6-30%, typically 10-20% CBD, e.g. about 18% CBD, and up to 5%, typically 0.5-3%, more typically 1-2% each of tetrahydrocannabinol (THC) and cannabinol (CBN). For example, medical grade CBD oil as provided by the Israel Ministry of Health contains about 10-30% CBD, in particular an average of 10%, 15%, 20% or 24% CBD, depending on the strain (wherein the initial extract may be diluted e.g. in olive oil or other pharmaceutically acceptable oils to obtain the determined concentration).

According to some embodiments, the composition of the invention may include about 1-30% CBD oil by weight. According to some embodiments, the composition of the invention may include about 2-25% CBD oil by weight. According to some embodiments, the composition of the invention may include about 5-15% CBD oil by weight. According to some embodiments, the composition of the invention may include about 7.5-12.5% CBD oil by weight. According to some embodiments, the composition of the invention may include about 8-12% CBD oil by weight. According to some embodiments, the composition of the invention may include about 9-11% CBD oil by weight. In some exemplary embodiments, the composition of the invention may include about 10% CBD oil by weight. In some embodiments, the content of CBD oil in the composition may be replaced by statement of CBD in the composition. Thus, in some embodiments, the composition comprises from about 2 to about 24% by weight of CBD, from about 6 to about 60% by weight of Bacopa monnieri extract, from about 6 to about 60% by weight of Centella Asiatica extract, and from about 5 to about 60% by weight of Withania Somnifera extract.

In some embodiments, one or more cannabinoids may be added to the composition in purified form. For example, CBD-containing oil may be manufactured synthetically, by addition of CBD (and optionally other cannabinoids and/or Cannabis-derived compounds such as terpenes) to oil, which may subsequently be used in manufacturing the composition.

According to embodiments of the invention, the composition may further include Bacopa monnieri extract. Typically, Bacopa monnieri extract may be obtained/extracted from the leaves. According to some embodiments of the invention, the composition may include about 5-60%, about 10-50%, or about 15-40% Bacopa monnieri extract, e.g. about 30% by weight Bacopa monnieri extract.

According to embodiments of the invention, the composition may further include Centella Asiatica extract. Typically, Centella Asiatica extract may be obtained/extracted from the leaves. According to some embodiments of the invention, the composition may include about 5-60%, about 10-50%, or about 15-40% Centella Asiatica, e.g. about 30% by weight Centella Asiatica extract.

According to embodiments of the invention, the composition may further include Withania Somnifera extract. Typically, Withania Somnifera extract may be obtained/extracted from the roots. According to some embodiments of the invention, the composition may include about 5-60%, about 10-50%, or about 15-40%, e.g. about 30% by weight Withania Somnifera extract.

According to some embodiments, there is thus provided a composition comprising about 5-25% by weight of CBD oil, about 15-40% by weight of Bacopa monnieri extract, about 15-40% by weight of Centella Asiatica extract, and about 15-40% by weight of Withania Somnifera extract. According to some embodiments, the composition may include about 8-15% by weight of CBD oil. In some embodiments, the composition may include about 10% by weight of CBD oil. According to some embodiments, the CBD oil may include about 10-30% by weight of CBD.

According to some embodiments, the CBD oil may be obtained from plant extract, or may be prepared synthetically.

In some embodiments, the composition may include about 30% by weight of Centella Asiatica extract. In some embodiments, the composition may include about 30% by weight of Bacopa monnieri extract. In some embodiments, the composition may include about 30% by weight of Withania Somnifera extract.

According to some embodiments, the composition comprises an orally-acceptable carrier.

In some embodiments, the composition is a pharmaceutical composition comprising one or more pharmaceutically acceptable excipients and/or carriers.

In some embodiments, the composition or the pharmaceutical composition is formulated for systemic administration. In some embodiments, the systemic administration is oral administration. In some embodiments, the formulation is in the form of liquid, an oily liquid form, drops, syrup, tablet, or capsule.

According to some embodiments, the composition of the present invention, e.g. a pharmaceutical composition, is for use in treating a mental disorder. According to one embodiment, the mental disorder is selected from Attention Deficit Hyperactivity Deficiency (ADHD) and post traumatic stress disorder (PTSD).

According to another aspect, the present invention provides a method for treating a mental disorder in a subject in need thereof, the method comprising administering to the subject a composition comprising from about 5 to about 25% by weight of CBD oil, about 15-40% by weight of Bacopa monnieri extract, about 15-40% by weight of Centella Asiatica extract, and about 15-40% by weight of Withania Somnifera extract, to thereby treat the subject. According to some embodiments, the mental disorder is selected from ADHD and PTSD.

Other objects, features and advantages of the present invention will become clear from the following description.

### DETAILED DESCRIPTION OF THE INVENTION

According to some embodiments, there are provided compositions and methods for treating mental disorders such as Attention Deficit Hyperactivity Deficiency (ADHD) and post traumatic stress disorder (PTSD), by utilizing a composition which includes CBD oil and one or more additional ingredients such as plant(s) extracts. The compositions and methods disclosed herein are particularly efficient, useful and safe with diminished side effects, as demonstrated herein. Systemic administration of the compositions (such as by oral administration) result in an efficient and rapid improvement of ADHD and PTSD symptoms, by showing a synergistic effect.

According to one aspect, the present invention provides a composition comprising from about 1 to about 30% by weight of CBD oil, from about 5 to about 60% by weight of Bacopa monnieri extract, from about 5 to about 60% by weight of Centella Asiatica extract, and from about 5 to about 60% by weight of Withania Somnifera extract.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The term “cannabinoid” as used herein generally refers to one of a class of diverse chemical compounds that act on a cannabinoid receptor in cells that repress neurotransmitter release in the brain. The term “cannabinoid” as used herein further refers a chemical compounds that acts on cannabinoid receptors or has a structure similar to the structure of a compound acting on cannabinoid receptor in cells. Ligands for these receptor proteins include the endocannabinoids (produced naturally in the body by humans and animals), the phytocannabinoids (found in Cannabis and some other plants), and synthetic cannabinoids (manufactured artificially).

The term “extract” as used herein refers to a product prepared by extraction by physical means (e.g. by comminuting, pressing, heating, pulsed electric field assisted treatments, shear treatments and pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence, which is free of, or substantially free of solvent. The term extract may be a single extract obtained from a particular extraction step or series of extraction steps. Extract also may be a combination of extracts obtained from separate extraction steps or separate feedstocks. Such combined extracts are thus also encompassed by the term “extract”. Any method of extraction and any solvent is encompassed. The extract may be obtained from any part of the plant e.g. from leaves, flowers, stems, roots, fruits and seeds. The extract may be aqueous or oily. Commonly used solvents are water, ethanol, ethyl acetate, CO₂, methanol, acetone, and acetic acid. According to one embodiment, the solvent is ethanol. According to another embodiment, the extraction is by CO₂. In particular, the term “extract” refers to a liquid or semi-solid or resinous substance obtained through extraction from plants defined in the present application, i.e. extracts obtained from Cannabis plant e.g. CBD-rich strain, Bacopa monnieri, Centella Asiatica or Withania Somnifera. In some embodiments, the term refers to a mixture of liquid or semi-solid, resinous substances obtained through extraction from two or more different plans. In some embodiments, the term refers also to a compound purified from the extract. According to some embodiments, the term “extract” has the meaning of a mixture or combination of two or more extracts. The extract also may be formulated into a pharmaceutical composition or food product, as described further below.

The term “Cannabis extract” as used herein refers to one or more plant extracts from the Cannabis plant. A Cannabis extract contains, in addition to one or more cannabinoids, one or more non-cannabinoid components, which are co-extracted with the cannabinoids from the plant material. Their respective ranges in weight will vary according to the starting plant material and the extraction methodology used. Cannabinoid-containing plant extracts may be obtained by various means of extraction of Cannabis plant material. Such means include but are not limited to: supercritical or subcritical extraction with CO₂, extraction with hot or cold gas and extraction with solvents. In some embodiments, the term refers to a mixture of liquid or semi-solid, resinous substances obtained through extraction from two or more different Cannabis species. In some embodiments, the term refers also to a compound purified from the extract. The term “Cannabis plant” as used herein, refers to plants of the genus Cannabis, including but not limited to Cannabis sativa, Cannabis indica, and Cannabis ruderalis. According to one embodiment, the Cannabis plant is a CBD-rich strain of Cannabis plant.

The term “natural cannabinoid” as used herein generally refers to a cannabinoid, which can be found in, isolated from and/or extracted from a natural resource, such as plants. “Synthetic cannabinoids” are a class of chemicals that are different from the cannabinoids found e.g. in Cannabis but which also bind to cannabinoid receptors.

In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

In some embodiments, the pharmacologically active cannabinoid may be selected from the group consisting of cannabidiol (CBD), tetrahydrocannabinol, Δ9-tetrahydrocannabinol (THC), Δ8-tetrahydrocannabinol, standardized marijuana extracts, Δ8-tetrahydrocannabinol-DMH, Δ9-tetrahydrocannabinol propyl analogue (THCV), 11-hydroxy-tetrahydrocannabinol, 11-nor-9-carboxy-tetrahydrocannabinol, 5′-azido-.Δ8-tetrahydrocannabinol, AMG-1 (CAS Number 205746-46-9), AMG-3 (CAS Number 205746-46-9), AM-411 (CAS Number 212835-02-4), (−)-11-hydroxy-7′-isothiocyanato-48-THC (AM-708), (−)-11-hydroxy-7′-azido-48-THC (AM-836), AM-855 (CAS Number 249888-50-4), AM-919 (CAS Number 164228-46-0), AM926, AM-938 (CAS Number 303113-08-8), cannabidiol (CBD), cannabidiol propyl analogue (CBDV), cannabinol (CBN), cannabichromene, cannabichromene propyl analogue, cannabigerol, CP 47,497 (CAS Number (1S,3R): 114753-51-4), CP 55,940 (CAS Number 83002-04-4), CP 55,244 (CAS Number 79678-32-3), CT-3 (ajulemic acid), dimethylheptyl HHC, HU-210 (1,1-Dimethylheptyl-11-hydroxy-tetrahydrocannabinol), HU-211 (CAS Number 112924-45-5), HU-308 (CAS Number 1220887-84-2), WIN 55212-2 (CAS Number 131543-22-1), desacetyl-L-nantradol, dexanabinol, JWH-051 (Formula C25H38O2), levonantradol, L-759633 (Formula C26H40O2), nabilone, O-1184, and mixtures thereof. Each possibility is a separate embodiment.

The terms “cannabidiol” and “CBD” are interchangeably used herein and refer to a non-psychotropic cannabinoid having structure as described in Formula I below, salt or derivatives thereof, such as Δ4-cannabidiol, Δ5-cannabidiol, Δ6-cannabidiol, Δ1,7-cannabidiol, Δ1-cannabidiol Δ2-cannabidiol Δ3-cannabidiol.

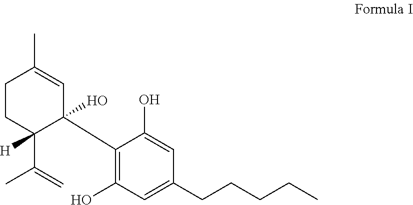 [SMILES: C=C(C)[C@@H]1CCC(C)=C[C@@]1(O)c1c(O)cc(CCCCC)cc1O]

As used herein the term “CBD oil” is directed to a lipophilic plant extract which comprises CBD and optionally one or more additional cannabinoids, such as, for example, but not limited to: THC, CBN, and the like. The term CBD Oil further encompasses oil formulations including pure cannabinoids (for example, synthetic forms) including CBD and optionally one or more additional cannabinoids. According to some embodiments, the term “CBD oil” encompasses also crude extract of Cannabis, in particular from a CBD rich strain, a fraction of the Cannabis extract comprising high concentration of CBD, or purified CBD. According to some embodiments, the CBD oil comprises less than 5 wt %, less than 3 wt %, less than 2 wt %, 1 wt %, less than 0.5 wt %, less than 0.2 wt % or less than 0.1 wt % of THC. According to some embodiments, the CBD oil comprises from 1 to 3.5, from 1.5 to 3.5 wt % or from 2 to 3 wt % of THC. According to one embodiment, the CBD oil comprises from 55 to 65 wt % of CBD and or from 2 to 3 wt % of THC. According to some such embodiments, the CBD:THC weight ratio in the CBD oil is at least 10:1, at least 15:1 or at least 20:1. According to some embodiments, the CBD:THC weight ratio is from 20:1 to 1:1 or from 20:1 to 1:1.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The term “excipient” as used herein has its conventional meaning and refers to any acceptable ingredient, which is commonly used in the pharmaceutical technology for preparing a granulate, solid or liquid oral dosage formulation, in food or food additive preparations.

The term “pharmaceutically acceptable carrier” or “pharmaceutically acceptable excipient” includes any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The term “carrier” refers to a diluent, adjuvant, excipient, or vehicle with which the therapeutic is administered. Such carriers can be sterile liquids, such as water or oils, including those of petroleum, animal, vegetable or synthetic origin, such as peanut oil, soybean oil, mineral oil, sesame oil and the like, polyethylene glycols, glycerin, propylene glycol or other synthetic solvents. According to some embodiments, the carrier is a pharmaceutically acceptable carrier.

The term “orally-acceptable carrier” as used herein refers to any physiologically acceptable carrier suitable and safe for oral administration to mammals and in particular to human. According to some embodiments, the orally-acceptable carrier is generally recognized as safe (GRAS).

The term “dosage form” denotes any form of the formulation that contains an amount of a cannabinoid or of a mixture of cannabinoids sufficient to achieve at least a partial therapeutic effect when appropriately administered. In some embodiments, the dosage form is for systemic, enteral, oral administration, including, but not limited to: oily liquid, liquid, drops, syrup, tablets, capsules, sustained release tablets and capsules, powders, granules, oral dispersible tablets, sublingual tablets or combinations thereof.

The terms “treat,” “treating,” or “treatment” as used herein, include reducing, alleviating, ablating, ameliorating, relieving, or lessening a symptom associated with either a chronic or an acute therapeutic scenario treatable with the composition of the invention. In some embodiments, the term includes, but is not limited to, alleviation or amelioration of one or more symptoms or parameters associated with a mental disorder such as ADHD or PTSD, such as improvement in parameters as assessed by various rating scales, tests, appearance and/or indices. In some embodiments, the term relates to: reducing, ameliorating and/or eliminating the ADHD or PTSD condition and/or the symptoms associated therewith.

As used herein, the term “mental disorder” and “mental illness” refer to those provided in the Diagnostic and Statistical Manual (DSM IV), American Psychological Association (APA). The terms include attention deficit hyperactivity disorder and post-traumatic stress disorder.

The terms “Attention Deficit Hyperactivity Disorder” and “ADHD” are used herein interchangeably and refer to a mental disorder of the neurodevelopmental type, in more detail, one of developmental disorders, which is characterized by inattention, hyperactivity, and impulsiveness. It is characterized by problems paying attention, excessive activity, or difficulty controlling behavior. The term includes all subtypes of ADHD (predominantly inattentive (ADHD-PI or ADHD-I), predominantly hyperactive-impulsive (ADHD-PH or ADHD-HI), and combined type (ADHD-C)). For purposes of the present invention, the term “symptoms of ADHD” refer to symptoms defined by the various DSM and ICD versions as well as other similar definitions of ADHD described above. Improvement of ADHD includes one or more of inhibition of aggression, and the specific behavior disorder of aggression includes aggression against self and/or others, hostility, hyperactivity, severe impulsiveness, etc. The improvement of impairment in continuous concentration, the improvement of a diminished rapid-reaction, and the improvement of reduced activity, which are examples of the improvement of attention function in ADHD of the present invention, can be evaluated by visual/perceptual seeking and/or systematic/continuous listening as a marker of the improvement. Improvement of one of the above mentioned symptoms or of symptoms defined by the various DSM and ICD versions is encompassed by the term treating ADHD.

The terms “Post-Traumatic Stress Disorder” and “PTSD” are used herein interchangeably and refer to a psychiatric condition in its broadest sense, as defined in DSM-IV-TR. The DSM-IV-TR defines “Post-Traumatic Stress Disorder” as characterized by persistent re-experiencing of an extreme traumatic event. The DSM-IV-TR sets forth a generally accepted standard for diagnosing and categorizing Post-Traumatic Stress Disorder.

The term “about” as used herein refers to any value, which lies within a range of ±10% of original value. The terms “% by weight” and “wt %” are used herein interchangeably and have the meaning commonly used in the field of pharmaceutical preparations. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In some embodiments, the cannabinoid is a natural cannabinoid. In some embodiments, the cannabinoid is a natural cannabinoid found in a Cannabis plant. In some embodiments, the cannabinoid is a synthetic cannabinoid. In some embodiments, the cannabinoid is a mixture of natural cannabinoids. In some embodiments, the cannabinoid is a mixture of synthetic cannabinoids. In some embodiments, the cannabinoid is a mixture of natural and synthetic cannabinoids.

In some embodiments, the CBD oil may include from about 2% to about 90% CBD by weight. In some embodiments, the CBD oil may include from about 2% to about 75% CBD by weight. In some embodiments, the CBD oil may include from about 2% to about 60% CBD, by weight. In some embodiments, the CBD oil may include from about 2% to about 50% CBD. In some embodiments, the CBD oil may include from about 2% to about 40% CBD by weight. In some embodiments, the CBD oil may include from about 2% to about 30% CBD by weight. In some embodiments, the CBD oil may include from about 2% to about 20% CBD by weight. In some embodiments, the CBD oil may include from about 2% to about 15% CBD by weight. In some embodiments, the CBD oil may include from about 5% to about 75% CBD by weight. In some embodiments, the CBD oil may include from about 5% to about 50% CBD by weight. In some embodiments, the CBD oil may include from about 5% to about 30% CBD by weight. In some embodiments, the CBD oil may include from about 10% to about 30% CBD by weight. In some embodiments, the CBD oil may include from about 5% to about 20% CBD by weight. In some embodiments, the CBD oil may include from about 7.5% to about 25% CBD by weight. In some embodiments, the CBD oil may include from about 7.5% to about 20% CBD by weight. In some embodiments, the CBD oil may include from about 7.5% to about 15% CBD by weight. In some embodiments, the CBD oil may include from about 10% to about 20% CBD by weight. In some embodiments, the CBD oil may include from about 20% to about 40% CBD by weight. In some embodiments, the CBD oil may include from about 30% to about 60% CBD by weight. In some embodiments, the CBD oil may include from about 55% to about 75% CBD by weight. In some embodiments, the CBD oil may include from about 55% to about 70% CBD by weight. In some embodiments, the CBD oil may include from about 55% to about 65% CBD by weight. In some embodiments, the CBD oil may include from about 60% to about 75% CBD by weight. In some embodiments, the CBD oil may include from about 60% to about 70% CBD by weight. In some embodiments, the CBD oil may include from about 65% to about 75% CBD by weight. According to some embodiments, the CBD oil comprises from 55 to 65 wt % of CBD. According to some embodiments, the CBD oil comprises less than 5 wt %, less than 3 wt %, less than 2 wt %, 1 wt %, less than 0.5 wt %, less than 0.2 wt % or less than 0.1 wt % of THC. According to some embodiments, the CBD oil comprises from 1 to 3.5, from 1.5 to 3.5 wt % or from 2 to 3 wt % of THC. According to one embodiment, the CBD oil comprises from 55 to 65 wt % of CBD and or from 2 to 3 wt % of THC. According to some such embodiments, the CBD:THC weight ratio in the CBD oil is at least 10:1, at least 15:1 or at least 20:1. According to some embodiments, the CBD:THC weight ratio is from 20:1 to 1:1 or from 20:1 to 1:1.

In some embodiments, the natural cannabinoid such as CBD is derived or isolated from an extract of a Cannabis plant. In some embodiments of the composition, the composition further comprises natural terpene(s) derived or isolated from an extract of a Cannabis plant.

In some embodiments, the pharmaceutical composition or the CBD oil may further include one or more cannabinoids in addition to CBD. In some embodiments, the one or more cannabinoids are selected from the group consisting of Cannabidiolic acid (CBDA), Tetrahydrocannabinol (THC), Tetrahydrocannabinolic acid (THCA), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM), salts thereof, and derivatives thereof.

In some embodiments, the composition or the CBD oil comprises about 30% to about 80% by weight of a cannabinoid or a mixture of cannabinoids. In certain embodiments, the composition or the CBD oil comprises about 40% to about 70% by weight of a cannabinoid or a mixture of cannabinoids. In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and mixtures of cannabinoids.

In some embodiments, the amounts of the active ingredients may be determined by the skilled artisan accordingly to meet the desired concentration in the final product. Thus, for example, the concentration of CBD (e.g. in the oil used as a starting material) may be determined such that the final composition may contain about 0.5-10%, 0.5-8%, 1-5%, 1-3%, 3-9%, 4-8% e.g. 1.8% or 5% by weight CBD. According to some embodiments, the composition comprises from 2 to 9 wt %, from 2 to 8 wt %, from 3 to 8 wt %, from 3 to 7 wt % or from 4 to 6 wt % of CBD. According to other embodiments, the composition comprises from 2 to 20 wt %, from 3 to 19 wt %, from 4 to 15 wt % or from 5 to 14 wt % of CBD. According to some embodiments, the composition comprises from 3 to 15, from 4 to 12 wt % or from 5 to 10 wt % of CBD. According to certain embodiments, the composition comprises 4.5, 5, 5.5, 6, 6.5, 7 or 7.5 wt % CBD.

According to some embodiments, the composition of the invention may include about 1-30% CBD oil by weight. According to some embodiments, the composition of the invention may include about 2-25% CBD oil by weight. According to some embodiments, the composition of the invention may include about 5-25% CBD oil by weight. According to other embodiments, the composition of the invention may include about 5-22% CBD oil by weight. According to some embodiments, the composition of the invention may include about 5-20% CBD oil by weight. According to some embodiments, the composition of the invention may include about 7.5-20% CBD oil by weight. According to some embodiments, the composition of the invention may include about 8-15% CBD oil by weight. According to some embodiments, the composition of the invention may include about 9-12% CBD oil by weight. In some exemplary embodiments, the composition of the invention may include about 10% CBD oil by weight. In some exemplary embodiments, the composition of the invention may include about 12% CBD oil by weight. According to some embodiments, the composition comprises from 3 to 9 wt %, from 8-20%, from 8-15 wt % or 8%, 9%, 10%, 11%, 12%, 13%, 14%, 15%, 16%, 17%, 18%, 19%, 20%, 21%, 22%, 23% or 24% by weight of CBD oil. According to one embodiment, the CBD oil comprises from 55 to 75 wt % or from 60 to 70 wt % CBD. According to some embodiments, CBD oil comprises from 55 to 65 wt %, CBD.

In some embodiments, the composition comprises at least about 20 mg of a cannabinoid or a mixture of cannabinoids, per 1 gr of composition. In some embodiments, the composition comprises at least about 50 mg of a cannabinoid or a mixture of cannabinoids, per 1 gr of composition. In some embodiments, the composition comprises about 20 mg to about 800 mg of a cannabinoid or a mixture of cannabinoids, per 1 gr of composition. In some embodiments, the composition comprises about 50 mg to about 500 mg of a cannabinoid or a mixture of cannabinoids, per 1 gr of composition. In some embodiments, the composition comprises at least about 20-500 mg of a cannabinoid or a mixture of cannabinoids, per 1 gr of composition. In some embodiments, the composition comprises at least about 30-150 mg or 40 to 120 mg of a cannabinoid or a mixture of cannabinoids, per 1 gr of composition. In some embodiments, the composition comprises at least about 40-90 mg of a cannabinoid or a mixture of cannabinoids, per 1 gr of composition. In some embodiments, the composition comprises at least about 50-70 mg of a cannabinoid or a mixture of cannabinoids, per 1 gr of composition. In some embodiments, the composition comprises about 20 mg, 50 mg, 100 mg, about 200 mg, about 300 mg, about 400 mg, about 500 mg or about 600 mg of a cannabinoid or a mixture of cannabinoids, per 1 gr of composition.

In some embodiments, one or more cannabinoids may be added to the composition in purified form. For example, CBD-containing oil may be manufactured synthetically, by addition of CBD (and optionally other cannabinoids and/or Cannabis-derived compounds such as terpenes) to oil, which may subsequently be used in manufacturing the composition.

According to embodiments of the invention, the composition may further include Bacopa monnieri extract. Typically, Bacopa monnieri extract may be obtained/extracted from the leaves. Bacopa monnieri extract has been known to improving cognitive activity (nootropic), act as a neuroprotective, antidepressant, sedative, anxiolytic, digestive and hepatic tonic, cholorectic and cholagogue, hepatoprotective and antiallergenic. According to some embodiments, the composition may include about 5-60% by weight of Bacopa monnieri extract. According to some embodiments, the composition may include about 10-50% by weight of Bacopa monnieri extract. According to some embodiments, the composition may include about 15-40% by weight of Bacopa monnieri extract. According to some embodiments, the composition comprises from 10 to 50 wt %, from 15 to 45 wt %, from 20 to 40 wt %, or from 15 to 35 wt % of Bacopa monnieri extract. According to some embodiments, the composition comprises from 20 to 50 wt % or from 25 to 45 wt % of Bacopa monnieri extract. According to some embodiments, the composition may include from about 25 to about 35 wt % of Bacopa monnieri extract. According to some embodiments, the composition may include about 30% by weight Bacopa monnieri extract.

According to embodiments of the invention, the composition may further include Centella Asiatica extract. Typically, Centella Asiatica extract may be obtained/extracted from the leaves. Centella Asiatica extract has been known as an adaptogen (promoting stabilization of physiological processes and of homeostasis), supporting the nervous system, aiding in tissue restoration, improving cognitive function, and functioning as an anxiolytic and an antioxidant. According to some embodiments, the composition may include about 5-60% by weight of Centella Asiatica extract. According to some embodiments, the composition may include about 10-50% by weight of Centella Asiatica extract. According to some embodiments, the composition may include about 15-40% by weight of Centella Asiatica extract. According to some embodiments, the composition comprises from 10 to 50 wt %, from 15 to 45 wt %, from 20 to 40 wt %, or from 15 to 35 wt % of Centella Asiatica extract. According to some embodiments, the composition may include from about 25 to about 35 wt % of Centella Asiatica extract. According to some embodiments, the composition comprises from 20 to 50 wt % or from 25 to 45 wt % of Centella Asiatica extract. According to some embodiments, the composition may include about 30% by weight Centella Asiatica extract.

According to embodiments of the invention, the composition may further include Withania Somnifera extract. Typically, Withania Somnifera extract may be obtained/extracted from the roots. Withania Somnifera extract has been known as an adaptogen, sedative, anti-inflammatory, antidepressant, immunoregulator and immunostimulator, nervine tonic, digestive stimulant and anti-proliferative. According to some embodiments, the composition may include about 5-60% by weight of Withania Somnifera extract. According to some embodiments, the composition may include about 10-50% by weight of Withania Somnifera extract. According to some embodiments, the composition may include about 15-40% by weight of Withania Somnifera extract tract. According to some embodiments, the composition comprises from 10 to 50 wt %, from 15 to 45 wt %, from 20 to 40 wt %, or from 15 to 35 wt % of Withania Somnifera extract. According to some embodiments, the composition comprises from 20 to 50 wt % or from 25 to 45 wt % of Withania Somnifera extract. According to some embodiments, the composition may include from about 25 to about 35 wt % of Withania Somnifera extract. According to some embodiments, the composition may include about 30% by weight Withania Somnifera extract.

According to some embodiments, there is provided a composition comprising from about 5 to about 25% by weight of CBD oil, from about 15 to about 40% by weight of Bacopa monnieri extract, from about 15 to about 40% by weight of Centella Asiatica extract, and from about 15 to about 40% by weight of Withania Somnifera extract. According to one embodiment, the present invention provides a composition comprising from about 5 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract. According to one embodiment, the present invention provides a composition comprising from about 5 to about 22% by weight of CBD oil, from about 20 to about 35% by weight of Bacopa monnieri extract, from about 20 to about 35% by weight of Centella Asiatica extract, and from about 20 to about 35% by weight of Withania Somnifera extract. According to one embodiment, the present invention provides a composition comprising from about 8 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract. According to one embodiment, the present invention provides a composition comprising from about 8 to about 22% by weight of CBD oil, from about 20 to about 35% by weight of Bacopa monnieri extract, from about 20 to about 35% by weight of Centella Asiatica extract, and from about 20 to about 35% by weight of Withania Somnifera extract. According to some embodiments, the CBD oil comprises from 40 to 80 wt % or from 30 to 60 wt % of CBD. According to one embodiment, the CBD oil comprises from 55 to 75 wt % or from 60 to 70 wt % CBD. According to another embodiment, the composition comprises from 2 to 12 wt % of CBD. According to another embodiment, the composition comprises from 4 to 11 wt % of CBD.

The term “comprising” has also the meaning of consisting. Thus, in some embodiments, the only extracts included in the composition are extracts of Bacopa monnieri, CBD rich Cannabis strain, Centella Asiatica and Withania Somnifera. According to such embodiments, the composition may further comprise non active excipient(s) and carrier(s). Thus in one embodiment, the composition consists of as active ingredients: CBD oil, Bacopa monnieri extract, Centella Asiatica extract and Withania Somnifera extract, each at concentrations as defined in the application, and optionally a non-active orally acceptable or pharmaceutically acceptable carrier and/or excipients.

According to some embodiments, there is provided a composition comprising therapeutically effective amounts of one or more of:

a) Cannabis, in the form of CBD oil;
b) Bacopa monnieri extract;
c) Centella Asiatica extract; and
d) Withania Somnifera extract.

Each possibility is a separate embodiment.

a) In some embodiments there is provided a comprising:
b) 1-30% by weight CBD oil or at least one cannabinoid;
c) 7.5-60% by weight Bacopa monnieri extract;
d) 7.5-60% by weight Centella Asiatica extract; and
e) 7.5-60% by weight Withania Somnifera extract.

Each possibility is a separate embodiment.

According to some embodiments, there is provided a composition including about 5-25% by weight of CBD oil, about 10-50% by weight of Bacopa monnieri extract oil, about 10-50% by weight of Centella Asiatica extract and about 10-50% by weight of Withania Somnifera extract. Each possibility is a separate embodiment.

The composition according to any one of the above embodiments may further comprise an orally acceptable carrier. According to some embodiments, the orally acceptable carrier is any carrier suitable for administration to a human being.

According to some embodiments, the composition is a pharmaceutical composition further comprising a pharmaceutically acceptable excipient and/or carrier.

According to some embodiments, the composition, e.g. a pharmaceutical composition is formulated for a systemic administration. According to one embodiment, the systemic administration is an oral administration. According to some embodiments, the composition is in the form of drops, liquid, oily liquid, capsules and tablets. According to some embodiments, the composition is formulated for oromucosal administration, e.g. for sublingual administration.

According to another aspect, the composition of the present invention is for use in treating a mental disorder. Thus, according to some embodiments, the composition according to any one of the above aspects and embodiments is for use in treating a mental disorder. According to one embodiment, the mental disorder is Attention Deficit Attention Deficit Hyperactivity Disorder (ADHD). According to another embodiment, the mental disorder is a post traumatic stress disorder (PTSD). Thus, according to one embodiment, the present provides a composition comprising from about 1 to about 30% by weight of CBD oil, from about 5 to about 60% by weight of Bacopa monnieri extract, from about 5 to about 60% by weight of Centella Asiatica extract, and from about 5 to about 60% by weight of Withania Somnifera extract, for use in treating a mental disorder selected from ADHD and PTSD. According to one embodiment, the composition is a pharmaceutical composition.

According to some embodiments, the composition for use comprises from 5 to 25 wt % or from 8 to 22 wt % or from 8 to 20 wt % CBD oil. According to some embodiments, the CBD oil comprises from 5 to 50 wt % of CBD. According to some embodiments, the CBD oil comprises from 40 to 80 wt % of CBD. According to some embodiments, the CBD oil comprises from 55 to 75 wt % of CBD. According to some embodiments, the CBD oil comprises from 30 to 60 wt % of CBD. According to some embodiments, the CBD oil comprises from 55 to 65 wt % of CBD. According to some embodiments, the composition, such as a pharmaceutical composition, comprises from 1 to 10 or from 2 to 8 wt % CBD. According to some embodiments, the composition, such as a pharmaceutical composition, comprises from 4 to 8 wt % CBD. According to some embodiments, the composition comprises from 10 to 50 wt %, from 15 to 45 wt %, from 20 to 40 wt %, or from 25 to 35 wt % of Bacopa monnieri extract. According to some embodiments, the composition comprises from 10 to 50 wt %, from 15 to 45 wt %, from 20 to 40 wt %, or from 25 to 35 wt % of Centella Asiatica extract. According to some embodiments, the composition comprises from 10 to 50 wt %, from 15 to 45 wt %, from 20 to 40 wt %, or from 25 to 35 wt % of Withania Somnifera extract.

According to one embodiment, the present invention provides a composition comprising from about 5 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract, for use in treating an ADHD. According to one embodiment, the present invention provides a composition comprising from about 5 to about 22% by weight of CBD oil, from about 20 to about 35% by weight of Bacopa monnieri extract, from about 20 to about 35% by weight of Centella Asiatica extract, and from about 20 to about 35% by weight of Withania Somnifera extract, for use in treating an ADHD. According to one embodiment, the present invention provides a composition comprising from about 8 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract, for use in treating an ADHD. According to one embodiment, the present invention provides a composition comprising from about 8 to about 22% by weight of CBD oil, from about 20 to about 35% by weight of Bacopa monnieri extract, from about 20 to about 35% by weight of Centella Asiatica extract, and from about 20 to about 35% by weight of Withania Somnifera extract, for use in treating an ADHD. According to some embodiments, the CBD oil comprises from 40 to 80 wt % or from 30 to 60 wt % or from 55 to 75 wt % of CBD. According to another embodiment, the composition comprises from 2 to 12 wt % of CBD. According to another embodiment, the composition comprises from 4 to 10 wt % of CBD.

According to one embodiment, the present invention provides a composition comprising from about 5 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract, for use in treating a PTSD. According to one embodiment, the present invention provides a composition comprising from about 5 to about 22% by weight of CBD oil, from about 20 to about 35% by weight of Bacopa monnieri extract, from about 20 to about 35% by weight of Centella Asiatica extract, and from about 20 to about 35% by weight of Withania Somnifera extract, for use in treating a PTSD. According to one embodiment, the present invention provides a composition comprising from about 8 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract, for use in treating a PTSD. According to one embodiment, the present invention provides a composition comprising from about 8 to about 22% by weight of CBD oil, from about 20 to about 35% by weight of Bacopa monnieri extract, from about 20 to about 35% by weight of Centella Asiatica extract, and from about 20 to about 35% by weight of Withania Somnifera extract, for use in treating a PTSD. According to some embodiments, the CBD oil comprises from 40 to 80 wt % or from 30 to 60 wt % or from 55 to 75 wt % of CBD. According to another embodiment, the composition comprises from 4 to 10 wt % of CBD.

According to some embodiments, the CBD oil comprises from 40 to 80 wt % or from 30 to 60 wt % of CBD. According to one embodiment, the CBD oil comprises from 55 to 75 wt % or from 60 to 70 wt % CBD. According to some embodiments, the CBD oil comprises from 55 to 70 wt % of CBD. According to some embodiments, the CBD oil comprises from 55 to 65 wt % of CBD. According to another embodiment, the composition comprises from 2 to 12 wt % of CBD or 4 to 10 wt % of CBD. According to one embodiment, the composition comprises from 4 to 8 wt % of CBD.

According to some embodiments, the composition is orally administered. According to one embodiment, the composition, e.g. a pharmaceutical composition is formulated as a liquid composition. According to other embodiments, the composition is formulated as capsules e.g. gel capsule. According to some embodiments, the composition is formulated as a tablet. According to another embodiment, the composition is formulated as a sublingual tablet.

According to some embodiments, the composition is a liquid composition administered in the amount of 1, 2, 3, 4, 5, 6, 7, 8, 9 or 10 drops 1, 2, 3, 4, 5 or 6 times a day. According to some embodiments, the composition is administered in the amount of 2, 3 or 4 drops 2, 3 or 4 times a day. According to one embodiment, the composition is administered to provide from 0.5 to 5 mg of CBD 2, 3, or 4 times a day. According to some embodiments, the composition is administered to provide from about 1 to about 3 mg of CBD 2, 3, or 4 times a day or from about 1.2 to about 2 mg of CBD 2, 3, or 4 times a day.

According to any one of the above embodiments, the composition of the present invention provides a synergistic anti-ADHD effect. According to another embodiment, the composition of the present invention provides a synergistic anti-PTSD effect.

According to some embodiments, the composition is co-administered with an active agent being a standard treatment of the disorder. According to one embodiment, the active agent is administered in a concentration or in a frequency lower than in the standard treatment.

According to another aspect, there is provided a method for treating a mental disorder. According to some embodiments, the mental disorder is selected from ADHD and PTSD. According to certain embodiments, the method provides treating a behavioral disease, disorder or condition in a subject in need thereof, comprising administering to the subject the composition of the invention. According to one embodiment, the behavioral disease, disorder or condition is selected from ADHD and PTSD. According to some embodiments, there is provided a method for treating ADHD in a subject in need thereof, comprising administering to the subject the composition of the invention. According to other embodiments, there is provided a method for treating PTSD in a subject in need thereof, comprising administering to the subject the composition of the invention. According to some embodiments, the method comprises administering the composition, e.g. a pharmaceutical composition of the present invention. According to some embodiments, the composition comprises a composition comprising from about 5 to about 25% by weight of CBD oil, from about 15 to about 50% by weight of Bacopa monnieri extract, from about 15 to about 50% by weight of Centella Asiatica extract, and from about 15 to about 50% by weight of Withania Somnifera extract.

In some embodiments, the composition or the pharmaceutical composition of the invention is formulated for systemic administration. In some embodiments, the composition or the pharmaceutical composition of the invention is formulated for oral administration. Formulations for oral administration include oily liquid, liquid, drops, tablets, capsules, and the like. Exemplary suitable carriers include vegetable or mineral oils, white petrolatum, branched chain fats or oils, animal fats and waxes. Stabilizers, humectants, preservatives and antioxidants may also be included, as well as agents imparting color, fragrance and/or flavor, if desired.

In some embodiments, the composition or the pharmaceutical composition may be administered systemically, for example, by administering the formulation in the form of oral drops, 1-2 daily. Administering the formulation by enteral route may be performed by various suitable means, and suitable dosage forms, such as oral administration of liquids, drops, tablets, capsules, and the like, or any other suitable dosage form as known in the art.

In some embodiments, the compositions may conveniently be presented in unit dosage form, and may be prepared by any of the methods well-known in the art. The selected dosage form (for example, liquid, oily liquid, drops, tablets, capsules.), may depend upon the desired therapeutic effect, severity of the ADHD or PTSD, subtype of the ADHD, the duration of the treatment, short term treatment effect, extended treatment effect, long term treatment effect, and the like.

In some embodiments, the administration regime may be determined while taking into account the clinical condition of the subject, the severity of the ADHD or PTSD condition, the subtype of the condition, the scheduling of administration, the subject's age, gender, body weight and other factors relevant to clinicians of ordinary skill in the art.

In some embodiments, the compositions of the invention may be provided in a pack, a patch, or dispenser device, such as a kit. In addition, the kit may include one or more carrier(s) and/or excipients.

In some embodiments, the kit may include the composition of the invention and optionally one or more suitable excipients or carriers and instructions for use of the kit in the treatment of ADHD or PTSD. In some embodiments, the kit may include a dispenser, such as a suitable dropper, for orally administering the composition.

According to another aspect, the present invention provides a composition comprising from about 1 to about 30% by weight of CBD oil, from about 5 to about 60% by weight of Bacopa monnieri extract, from about 5 to about 60% by weight of Centella Asiatica extract, and from about 5 to about 60% by weight of Withania Somnifera extract, for preparation of a medicament for treating a mental disorder, e.g. ADHD or PTSD.

As used herein, the singular form “a,” “an,” and “the” include plural references unless the context clearly dictates otherwise. For example, the term “a compound” or “at least one compound” may include a plurality of compounds, including mixtures thereof.

The term “comprising” means that other steps and ingredients that do not affect the final result can be added. This term encompasses the terms “consisting of” and “consisting essentially of”.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention.

### Examples

### Example 1. Plant Extraction Process

The following extraction process was employed to obtain the following oil plant extracts:

1. Plant material was ground in the presence of ethanol to form a mixture of ground plant material and ethanol.

2. The resulting mixture was incubated for several days, depending on the type of plant and ambient temperature.

3. After incubation, the mixture was filtered.

4. After the filtration, ethanol was evaporated to receive concentrated extract.

5. For CBD oil only: the concentrated extract was heated in a vacuum oven in order to activate the molecule(s).

The homogenous obtained extracts, including CBD oil, were used for the preparation of the tested compositions. An exemplary CBD oil obtained from a particular Cannabis species comprises about 61.5 wt % of CBD (plus about 2 wt % CBDA). Additional components that were detected in the CBD oil were THC (about 2.5 wt %), and cannabichromene (about 1.8 wt %). It is clear that the content may vary due to different extraction conditions and Cannabis strains.

### Example 2. Compositions (Formulations) for Treatment of ADHD

Formulations for oral administration were prepared as described below, and their effect on ADHD was tested in subjects in need thereof, upon daily administration of 1-2 drops of a 1 ml formulation.

Formulation #1—a combination of CBD oil with Bacopa monnieri extract in 50/50 ratio. The results showed some improvement in level of attention and concertation. The treatment was successful for a small proportion of subjects, whereas some of the subjects reported an increase in headaches.

Formulation #2—a combination of 5% CBD oil with 50% Bacopa monnieri extract and addition of 45% Withania Somnifera root extract. The formulation was tested on 12 subjects (8 suffering from ADHD and 4 from PTSD) and the effect was assessed by feedback obtained from the subjects. The results showed that the formulation was better in reducing hyperactivity but still not all subjects reported a significant change in Attention-deficit.

In a further experiment, the concentration of CBD oil was increased to 10 wt % and the concentration of Withania Somnifera root extract was reduced to 40 wt % (Formulation 2A). The formulation was administered to the same patients who previously received Formulation 2. The patients reported an improvement of the ADHD symptoms such as suppression of surrounding noise but still reported disoriented thinking. As a next step, 30 wt % Centella Asiatica was added while maintaining 10 wt % of CBD oil. As a result, Formulation #3 was obtained.

Formulation #3—a combination of 10 wt % CBD oil with 30 wt % Bacopa monnieri extract, 30 wt % Withania Somnifera root extract and the addition of 30 wt % Centella Asiatica extract. The results obtained showed that for all subjects (the same 12 subjects previously administered with Formulation 2B) a significant improvement in the level of attention and concentration, reduction of hyperactivity was reported by all ADHD patients, with no side effects (such as headaches). The PTSD patients reported an improvement in their quality of life and reduction in stress.

CBD oil used in the above compositions contained 55-75 wt % of CBD. In some particular examples, CBD oil comprised about 61 wt % of CBD. The CBD oil contained also additional cannabinoids such as THC (about 2.5 wt %).

Thus, Formulation (Composition) #3 exhibited a synergistic effect on alleviating ADHD condition in treated subjects, without causing undesired side effects. Subjects that previously administered CBD in different formulation without significant improvement in ADHD symptoms reported much better effect of Formulation #3.

### Example 3. Modified Compositions for Treatment of ADHD

A 14 years old subject diagnosed as an ADHD patient who has been being treated with methylphenidate (Ritalin) started receiving a formulation comprising extract from Bacopa extract, Withania Somnifera root and Centella Asiatica. The subject gradually reduced the dose of Ritalin until complete cessation of using drug. The formulation had only a mild effect in comparison to previously used Ritalin. Therefore, the subject started taking Formulation #3 with 5 wt % of CBD oil (equivalent to about 2.5-4 wt % of pure CBD)—2 drops 2 times a day.

Compositions used in the present example are based on Formulation #3 of Example 2, in which the concentration of CBD oil was varied. The increase or decrease of the concentration of CBD oil was compensated by according increase or decrease of the concentrations of all other extracts. Such, composition comprising 5 wt % CBD further comprises 33.3 wt % of each of Bacopa extract, Withania Somnifera root and Centella Asiatica extracts. Composition comprising 12 wt % of CBD oil comprise 29.3 wt % of each of Bacopa extract, Withania Somnifera root and Centella Asiatica extracts. Composition comprising 25 wt % CBD comprises 25 wt % of each of Bacopa extract, Withania Somnifera root and Centella Asiatica extracts.

Three additional subjects suffering from ADHD started receiving the composition comprising 5 wt % CBD oil and extracts from Bacopa extract, Withania Somnifera root and Centella Asiatica (Formulation #3 with 5 wt % CBD oil).

Despite the fact that the patients reported an improvement in their general feeling and symptoms (after 2 months of administration), especially in reduction of white noise and in their ability to focus, the patients requested to increase the dose of CBD. Thus, after 2 months of administration of the composition comprising 5 wt % CBD oil, the concentration of CBD oil was increased to 25 wt % (equivalent to about 12-19 wt % of pure CBD) for all 4 patients. The patients reported that they were tired, less focused and 2 of them reported dizziness. Therefore, the concentration of CBD oil was reduced to 12 wt % (equivalent to about 7-9 wt % of pure CBD).

After one month, all ADHD patients reported a significant improvement in their cognition, writing ability, and focus. In several further trials to find the most effective range of concentrations of CBD oil, we found that below 8 wt % there was a very moderate effect and above 25 wt % of CBD oil, adverse effects appeared.

### Example 4. Modified Compositions for Treatment of PTSD

A patient diagnosed as having post-traumatic stress disorder (PTSD) who has been being treated with 30 mg/day of CBD started receiving a formulation comprising 5 wt % CBD oil and extracts from Bacopa extract, Withania Somnifera root and Centella Asiatica (formulation #3 with 5 wt % of CBD oil), 2 drops TID instead of the previous treatment. The formulation provided a pleasant but weak effect.

After 2 months of administration of the composition comprising 5 wt % CBD oil, the concentration of CBD oil was increased to 25 wt %. The patient reported tiredness and being less focused. Therefore, the concentration of CBD oil was reduced to 12 wt %. An additional PTSD patient started receiving the formulation with 12 wt % of CBD oil.

After one month, the patients reported a significant improvement in their quality of life, reduction in stress, improvement in sleep, relationship and in sex.

In several further trials to vary the CBD oil concentration, we found that below 8 wt % there was a very moderate effect and above 25 wt % of CBD oil, adverse effects appeared.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-16. (canceled)

17. A composition comprising from about 5 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract.

18. The composition according to claim 17, comprising from about 8 to about 20% by weight of CBD oil.

19. The composition according to claim 17, wherein said CBD oil comprises from about 55 to about 75% by weight of CBD.

20. The composition according to claim 17, wherein the CBD oil is obtained from plant extract, or is synthetically prepared.

21. The composition according to claim 17, comprising from about 25 to about 35% by weight of Bacopa monnieri extract.

22. The composition according to claim 17, comprising from about 25 to about 35% weight of Centella Asiatica extract.

23. The composition according to claim 17, comprising from about 25 to about 35% by weight of Withania Somnifera extract.

24. The composition according to claim 17, comprising from about 8 to about 22% by weight of CBD oil, from about 25 to about 35% by weight of Bacopa monnieri extract, from about 25 to about 35% by weight of Centella Asiatica extract, and about from about 25 to about 35% by weight of Withania Somnifera extract.

25. The composition according to claim 17, wherein the composition further comprises an orally acceptable carrier.

26. The composition according to claim 17, wherein the composition is a pharmaceutical composition comprising one or more pharmaceutically acceptable excipients.

27. The composition according to claim 17, formulated for a systemic administration.

28. The composition of claim 27, wherein the systemic administration is an oral administration.

29. The composition according to claim 27, wherein the composition is in the form of drops, liquid, oily liquid, capsules, or tablets.

30. A method for treating a mental disorder in a subject in need thereof, the method comprises administering to the subject a composition comprising from about 5 to about 22% by weight of CBD oil, from about 15 to about 40% by weight of Bacopa monnieri extract, from about 15 to about 40% by weight of Centella Asiatica extract, and from about 15 to about 40% by weight of Withania Somnifera extract, wherein the mental disorder is selected from the group consisting of Attention Deficit Hyperactivity Disorder (ADHD) and post traumatic stress disorder (PTSD).

31. A method for treating a mental disorder selected from the group consisting of Attention Deficit Hyperactivity Disorder (ADHD) and post traumatic stress disorder (PTSD) in a subject, comprising administering to the subject the composition according to claim 17.

32. The method according to claim 30, wherein the composition is orally administered.

33. The method according to claim 31, wherein the composition is orally administered.
